(19) United States
(12) Patent Application Publication
(10) Pub. No.: US 2017/0000388 A1
(43) Pub. Date: Jan. 5, 2017

(54) SYSTEM AND METHOD FOR MAPPING MOVING BODY PARTS

(71) Applicant: ICURA APS, Copenhagen (DK)

(72) Inventors: Lars JESSEN, Copenhagen K (DK); Jakob Mandoe NIELSEN, Bronshoj (DK); Steffen WINTHER, Copenhagen k (DK); Jesper Harding SORENSEN, Copenhagen k (DK)

(73) Assignee: Icura ApS, Copenhagen S. (DK)

(21) Appl. No.: 15/113,605

(22) PCT Filed: Jan. 9, 2015
(86) PCT No.: PCT/EP2015/050324

§ 371 Date: Jul. 22, 2016

(30) Foreign Application Priority Data
Jan. 24, 2014 (DK) PA 2014 70035

(51) Int. Cl. A61B 5/11; A61B 5/00

(52) U.S. Cl. CPC A61B 5/1127; A61B 5/0024; A61B 5/112; A61B 5/744; A61B 5/0022; A61B 5/486; A61B 5/7405; A61B 2560/0238; A61B 2505/09; A61B 2562/0219; A61B 2562/0223

(57) ABSTRACT

The present invention relates to a method for analysing movements of main body parts of a moving person, the method comprising the steps of attaching one or more sensors to selected main body parts, each sensor comprising means for wireless communication of data, calibrating data from the one or more sensors, and mapping the calibrated sensor data to a virtual 3D avatar. Moreover, the present invention relates to a system capable of performing the method.

### FIELD OF THE INVENTION

The present invention relates to a system involving a mobile motion sensor platform and an associated method for monitoring, rehabilitating, training and diagnosing patients, such as orthopaedic patients after surgery or patients having for example diabetic, heart and/or lung problems, cancer or other types of patients.

### BACKGROUND OF THE INVENTION

The prior art within this field is disclosed in details in the patent literature, such as in US 2008/0285805 and WO 2012/139868.

In short US 2008/0285805 discloses a system for capturing motion of a moving object via a plurality of motion sensor modules placed on various body segments. The sensor modules capture both 3D position and 3D orientation data relating to their respective body segments, thereby gathering motion data having six degrees of freedom with respect to a coordinate system not fixed to the body. Each body sensor collects 3D inertial sensor data and, optionally, magnetic field data. In embodiments, either DSP circuitry within the sensor modules or an external computing device, processes the sensor data to arrive at orientation and position estimates by using an estimation algorithm, such as a Kalman filter or a particle filter. The processing includes biomechanical model constraints that allow flexibility in the joints and provide characteristics for various joint types. To improve estimation accuracy, the system may be integrated with various types of aiding sensors.

WO 2012/139868 teaches a system and methods to perform rehabilitation or physical therapy exercise while doing specifically designed video-games with the support of a therapist. Patient plays said video-games with external controllers with motion sensors connected to a pc or a laptop. The therapist can influence a gaming session of the patient by setting on a shared web-service thresholds for the patient. Said settings are gathered before starting a gaming session and patient movements are filtered by said settings to control the video-game. The patient is then limited in the movements by the feedbacks provided by the audio-visual interface of the video-game: movements on the screen are a result of the real movement done by the patient with said motion sensors filtered by the settings imposed by the therapist on the shared web space. On the other side, a patient with problems in doing some movements, can effectively play a video-game thanks to filtering imposed by the therapist. Information about the game played, and consequently about movements performed, are finally uploaded on the web-service for further analysis by the therapist.

It is a disadvantage that the system disclosed in WO 2012/139868 requires the support of a therapist. The system uses video games, but it does not map the movements of the patient onto a 3D avatar. Whereas parameters in the video games can be adjusted, there is no system for adding new games.

US 2008/0285805 proposes a capture system rather than a real time system for training. Also, US 2008/0285805 does not propose the use of a 3D avatar and it does not specify a method for calibration that can be performed by patients themselves.

None of the above-mentioned prior art systems teaches using analysis and visualization for means of creating a better understanding of the patients own movements/quality of movements.

Moreover, none of the above-mentioned systems mention the use of specific training exercises, such as for example abduction exercises or standardized exercises including senior fitness test.

It may be seen as an object of embodiments of the present invention to provide a system for self-monitored training. The system allows patients to train unassisted of a therapist—for example at the patient's own home.

It may be seen as a further object of embodiments of the present invention to allow a therapist to change or modify training parameters remotely.

### DESCRIPTION OF THE INVENTION

The above-mentioned object is complied with by providing, in a first aspect, a method for analysing movements of main body parts of a moving person, the method comprising the steps of
attaching one or more sensors to selected main body parts, each sensor comprising means for wireless communication of data,
calibrating data from the one or more of sensors, and
mapping the calibrated sensor data onto a virtual 3D avatar.

It is advantageous of the present invention that the mapping of the calibrated sensor data onto the 3D avatar is performed real time, i.e. on the fly as the person actually moves his/hers body parts.

The method maps movement of the patient onto a 3D avatar to guide and increase body awareness during exercises and to further a better understanding of how to perform exercises correctly. Moreover, the proposed method is dynamic in terms of adding new exercises in that an exercise editor makes it is easy to add new exercises. Finally, different calibration methods are proposed that make it possible for the patient to calibrate the system unassisted for instance with a hands free calibration

The step of calibrating data may involve position calibration, calibration via exercise, dynamic calibration and/or hands free calibration via automated position calibration.

The present invention further relates to a step of making the calibrated sensor data available via a web service. Alternatively or in combination therewith calibrated sensor data may be stored or hosted in a number of the one or more sensors, or in association therewith, such as on a SD card insertable in at least one sensor.

In a second aspect the present invention relates to a use of the method according to the first aspect. Said use may involve creating and editing training exercises via an exercise editor.

In a third aspect the present invention relates to a mobile system for analysing movements of main body parts of a moving person, the mobile system comprising
one or more sensors adapted to be attached to selected main body parts of the moving person, each sensor comprising means for wireless communication of data,
processor means for calibrating data from the one or more sensors, and
means for mapping the calibrated sensor data to a virtual 3D avatar.

The mobile system may further comprise an accessible unit for hosting at least the calibrated sensor data. The accessible unit may be in communication with a web service.

Alternative, a number of the one or more sensors may be adapted to store or host calibrated sensor data for example on a SD card being insertable in at least one sensor.

The processor means may form part of a portable device, such as a mobile phone or a tablet.

In a fourth and final aspect the present invention relates to a computer program product for performing the method of the first aspect when said computer program product is run on a processor, such as a computer, mobile phone, tablet etc.

### BRIEF DESCRIPTION OF THE DRAWINGS

The present invention will now be explained with reference to the accompanying figures where

FIG. 1 shows the system topology,

FIG. 2 shows a typical motion sensor, and

FIG. 3 shows an example of the positioning of the motion sensors.

While the invention is susceptible to various modifications and alternative forms specific embodiments have been shown by way of examples in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

In its most general aspect the present invention relates to a real time system involving a mobile motion sensor platform and an associated method for monitoring, rehabilitating, training and diagnosing patients, such as orthopaedic patients after surgery or patients having for example diabetic, heart and/or lung problems, cancer or other types of patients.

The system and method apply real time information provided by one or more sensors positioned on one or more main body parts of the patient. In one embodiment a real time system applying only a single sensor for monitoring levels of activity and movement, and performing simple exercises, is provided.

The real time system of the present invention may be configured to analyze the quality of performed exercises and movements in relation to specified quality parameters, such as numerical angles, rotations for example in relation to flexion, elevation and anduction of the human limbs. Certain numerical thresholds are used for deeming the quality of an exercise.

Moreover, the real time system of the present invention may be configured for playing back audio messages in response to a quantity and a quality of movements or exercise performances. Even further the real time system of the present invention may be configured to provide visual feedback in response to movements and exercise performances. Even further, the real time system of the present invention may be configured to record movements and extracting certain algorithms for use in developing additional exercises.

The system according to the present invention may thus comprise:
one or more wireless motion sensor units. Each wireless motion sensor unit consists of an MCU (micro controller unit), a short range radio module, 3-axis accelerometer, 3-axis gyroscope, 3-axis magnetometer, rechargeable battery, and charging circuit. The charging can be done for example via USB or via induction.
a mobile processing device with a graphical user interface such as but not limited to a tablet, smartphone or computer. The processing device runs the software for calibration, training and monitoring. The device can provide real time feedback to the user
a server with a web service and a database.
web page interface.
API (application programming interface) for accessing the web service and database making it possible to create a range of applications in different programming languages and on different hardware platforms.
An exercise editor for creating and editing training exercises.

The one or more wireless motion sensors can measure 3D orientation. When the sensors are attached to the limbs of a person, the orientations of the sensors are mapped onto a virtual avatar (skeleton) in the application running on the processing device. This provides a real time representation of how the person moves. This allows the system to provide real time feedback to the person on certain movements or movement patterns. The movement data is stored on the processing device and is also uploaded to a web service. The data can then be accessed by other users for analysis and assessment.

The user of the system turns the sensors on and attaches the sensors to his/her limbs (as specified for the given type of training) with an elastic band or similar. The user then opens the application on the processing device and the sensors connect automatically. The application now receives motion readings from the sensors and the user performs a calibration process. After the calibration, the 3D orientation of each sensor is mapped onto an avatar. The application can provide real time feedback (audio and visual) on certain movements or on specific training exercises. The application stores the 3D orientation data along with any relevant training statistics. The data is uploaded regularly to the web service when the processing device has an available internet connection. Another user (for example a therapist or a doctor) can then access the data through a web page or an application which allows him/her to view the data graphically or play the motion sequence as a 3D animation. If the system is used for training or rehabilitation the user is also able to adjust exercises or make changes to the training program. Any changes made will be updated on the processing device via the internet connection.

Calibration

The system can be calibrated in a number of different ways depending on the purpose of monitoring/training and the sensor setup. Calibration is key to getting valid data from the monitoring/training.

Position Calibration:

This method takes the user through 2 or more positions. When in a specific position the user presses a button in the graphical interface to confirm and the system takes a snapshot of the actual sensor orientations in this position. Based on the data collected in the different positions, the limbs of the user are mapped to a 3D avatar representation in the application. The calibration system is based on a rigged 3D model, which makes it easy to deploy new calibration sequences depending on which part of the body should be monitored and to meet restricted movement requirements for some patients.

Automated Position Calibration:

This works like the position calibration described above. However, using the accelerometer of the motion sensor, a number of preset trigger points can be set. This allows the system to automatically detect when the patient is in the required position and thus does not require the user to press any buttons during the calibration process to confirm positions. After the position has been detected, the patient must stay still in that position for a few seconds for the calibration process to complete. This handsfree approach is especially useful when the system is used on the arms.

Calibration Via Exercise:

This method integrates warm up or a particular exercise with the calibration. During the exercise, the patient is instructed to perform certain movements. Based on the data collected from these movements the limbs of the user are mapped to a 3D avatar representation in the application. This method also offers a hands free approach and allows the calibration to be tightly integrated with the exercise experience.

In all of the above methods the quality of the calibration can be ensured both visual confirmation by the user, but also by the system, which can be set to only accept the calibration as valid if certain thresholds are met.

Dynamic Calibration:

After the initial calibration of the system, several factors can cause the calibration to become inaccurate or lose its validity—for instance if the sensors are moved or due to errors caused by magnetic disturbances. This can be mitigated in a number of ways: A biomechanical model can be employed to monitor if movements are registered to be outside the normal human range of movement, and the calibration can be offset accordingly. Calibration via exercise (see above) can also be employed during training, comparing the actual movements in a given exercise with the expected movements, which allows for ongoing adjustments of the calibration.

The calibration procedure is based on a probabilistic model, where each measurable quantity is assigned an expectation value and a variance (or other probability distribution) under the assumption that the calibration is correct. The calibration parameters are then optimized to maximize the probability of the actual measurements in the model, hence yielding the optimal calibration parameters from the observed measurements. The information encoded in the model includes biomechanical constraints, sensor placement constraints and, if applicable, expected positions and movements from position and exercise based calibration as described above.

In some embodiments of the system, there might not be an initial calibration, but the system will be calibrated solely via dynamic calibration.

Furthermore different kinds error correction methods can be employed to improve the data quality and user experience. For example by analysing the incoming data from the sensors the system can detect if the sensors have been placed on the correct limb, and if not adjust accordingly.

Exercise Training and Monitoring

The system can be used for exercise training and monitoring. In the following emphasis will be put on training purposes. However, monitoring of for example posture and a number of steps taken through a predetermined period are important for in particular very weak patients.

Each exercise consists of x number of positions. Using analyser modules, which measure for example the angle between two limbs or distance between two limbs, the system can determine real time whether the patient is within a given position or not. By subdividing the range of the analyser modules, a quality assessment can be made of how close to the ideal position the patient has come. If an exercise consists of a position A and B the system can count and keep track of repetitions for the patient by adding a repetition each time the patient has moved from A to B to A. Further relevant exercise parameters can be identified such as but not limited to stability or direction of a limb, acceleration and deceleration in an exercise, which can be determined as the speed between two positions.

Gait Analysis and Training

The system can be used for gait training and encouraging a patient to maintain a balanced walk with equal amount of weight on each leg. In order to reveal an asynchronous stride the system looks for “positive” step length. By positive step length we mean the extended forward position of the foot in relation to the upper body position. By comparing the positive step lengths for the left and right leg the system can determine how asynchronous the stride is. The balance in the stride can be shown visually to the patient in real time on the device. An audio message will alert the user if the stride has been asynchronous beyond a predefined limit for a certain amount of time. The audio interface makes the patient independent of a graphical interface and allows the patient to focus on their exercise while still getting feedback from the system if needed.

After the walk, statistics will be compiled comparing the positive step length for the left and right leg. From the compiled graphs further analysis can be made for example estimating the time when the patient becomes too tired to benefit from the exercise.

Exercise Editor

The training exercises are added to the system via the exercise editor. An exercise consists of a number of limb positions, quality parameters, and audio messages. The exercise editor has both a technical user interface and a user interface aimed at for example physiotherapists. The technical user interface makes it quick for persons with some technical knowledge of the system to create new exercises, but also allows for creating new types of parameters and exercise flows. Via a graphical interface the therapist, doctor or similar can choose from a bank of predefined positions or the therapist can record an exercise and extract key positions. Apart from positions the therapist can add relevant parameters for the exercise such as acceleration and deceleration. For positions and parameters the level of difficulty can be adjusted. Position keys and parameters can be linked to audio messages that will be played to assist the patient if he/she has difficulty reaching a position or complying with a parameter.

Existing exercises can be loaded into the editor and the therapist can edit positions, parameters and audio messages.

Sensor Management

The sensors are assigned and registered to a given processing device on the server. The sensors have unique identifiers managed centrally on the server. Administrative personnel such as therapists can replace a sensor with a new sensor via the web interface. After replacement the new sensor is referenced to the device and the device will receive the new sensor configuration the next time it has internet connection.

### Example I

### Self-Monitored Home Training (Rehabilitation)

In this embodiment the system is used for rehabilitation training in the patient's home. In this particular embodiment the system and method is used for training of knee and hip alloplastic patients, but the system can also be used on other areas of the body and for other diagnoses.

When the patient starts his/her training program. The physiotherapist adds the patient in the database via the online web interface and assigns a mobile device to the patient.

Based on the physiotherapist assessment of the patient's abilities the physiotherapist constructs a training program that fits the patient via the online interface. Premade templates (advanced, intermediate, beginner) make it easy for the therapist to create the program and then adjust it to fit the particular needs of the patient. The patient is given a mobile device (for example a smartphone or a tablet) with a training application and 5 sensors to take home.

The training application in this embodiment contains a training calendar, a knowledge bank and a chat functionality. The training calendar is where the patient can see today's exercises and start his/her training. It is also from the training calendar the patient can start training and see statistics on completed training. The knowledge bank contains information about the surgery/procedure, training, FAQs and other relevant (a web service allows the therapist to edit the content of the knowledge bank). The chat functionality allows the patient to chat to other patients following a similar program and to chat with the therapist. The app also provides contextual help (relevant information regarding the particular task that the patient is doing), which is accessed by pressing the help icon.

In this embodiment the training app allows the patient to perform two types of training: Exercises and gait training.

When the patient starts the training either exercises or gait training, instructions will guide the patient to apply the appropriate sensor to the appropriate limb using velcro straps. The patient will be guided through the calibration process (see Calibration). After the calibration the patient can move around to make a visual verification of the calibration by watching the 3D avatar on screen (which is mapped to the sensor movement), and the system will make a verification of the calibration as well using a biomechanical model. Upon a successful calibration the user starts the training:

Exercise Training:

A video or animation along with audio instructions will show the patient how to perform the exercise correctly. When performing the exercise, the patient will be guided by targets or pointers in the graphical user interface, and the repetitions will be counted automatically by the system (and is conveyed to the user both graphically and via audio). Audio messages related to measured parameters of the exercise will be played back to the user if necessary. The system will thus continuously monitor if the patient scores below the preset parameter threshold for a number of repetitions, the system will play the related audio message. The system is designed so the patient does not have to look at the screen once comfortable with the exercise, but can rely on the audio feedback, and thus free up mental capacity to concentrate on performing the exercise correctly. Each exercise will be scored based on the accuracy of the execution. In the training calendar the patient can follow the progress of the training.

Gait Training:

The gait training monitors how balanced the walk pattern of the patient is. It also tracks how far the patient walks each time via GPS (or other location services provided by the mobile device). Visual information about distance, pace, and balance is presented in the graphical interface. Audio messages relay the same information allowing the patient to perform the training with headphones and the phone in the pocket. Walk statistics will be saved in the training calendar, so the patient can follow the progress of the training. And the data will also be uploaded to the server.

Online Monitoring:

The physiotherapists can follow the progress of the training online as the training data is uploaded regularly. The physiotherapist can monitor both the quantity and quality of the training. If the proscribed quantity of training has not been met it will be indicated and if the quality is below a certain level this will also be indicated. The therapist can adjust the training program at any time adding or removing exercises, changing the difficulty or the frequency. The system automatically increases the difficulty of an exercise if the patient has performed well in that particular exercise over a period of time. This auto progression of exercises is designed to save time for the physiotherapist as the physiotherapist does not need to continuously monitor the difficulty levels of all exercises. However, the physiotherapist can always overrule the system and set the difficulty manually.

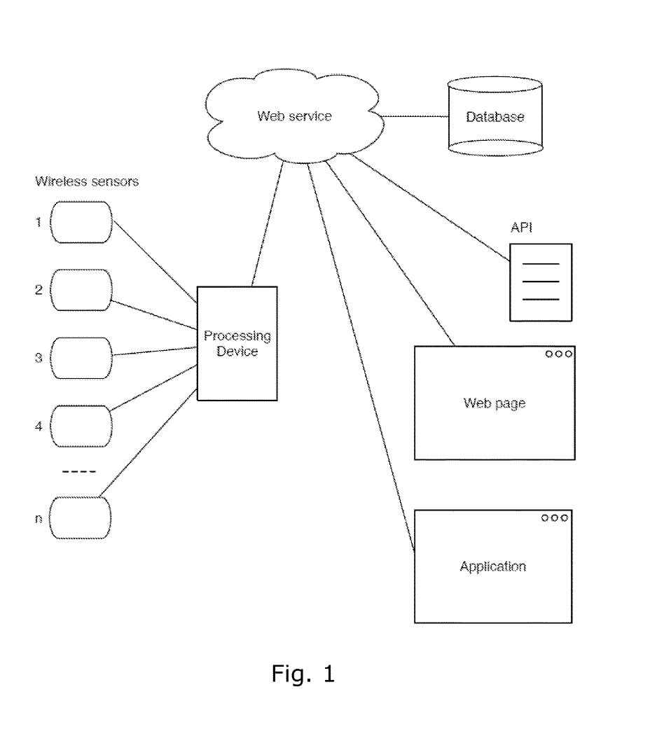

The system topology is depicted in FIG. 1. As seen the system comprises a number of motion sensors. The wireless sensors are in communication with a processing device which may be a computer, a tablet or a mobile phone. A web service and an associated database facilitates that data, such as calibrated motion data, may be accessed from a remote location via for example a web page. An API for accessing the web service and database makes it possible to create a range of applications in different programming languages and on different hardware platforms.

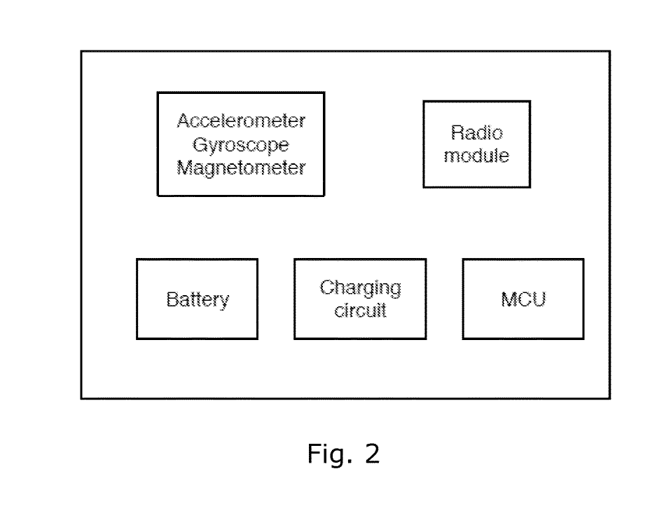

A typical motion sensor is shown in FIG. 2. As seen in FIG. 2 and as previously disclosed each sensor is preferably a wireless motion sensor unit consisting of an MCU (micro controller unit), a short range radio module, 3-axis accelerometer, 3-axis gyroscope, 3-axis magnetometer, rechargeable battery, and charging circuit. The charging can be done for example via USB or via induction.

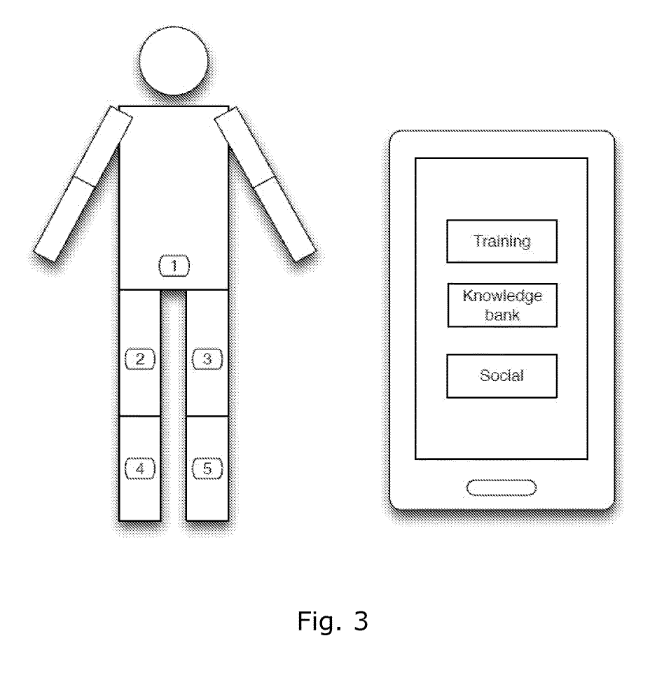

An example of the positioning of the motion sensors is illustrated in FIG. 3 where a total of five motion sensors have been attached to a person. The five sensors are in wireless communication with for example a mobile phone which acts as a data processing unit.

### Example II

### Attrition

In this embodiment the system is used to monitor attrition in work situations. The movement sensors are attached before the workday begins and a mobile processing device records the movement data during the workday. The data is sent to the server, processed, and statistics are produced on certain work positions to expose movement patterns that could lead to attrition such as repetitive movements. Based on the knowledge of potential harmful movement patterns the system can use pattern recognition on the processing device to alert the user of the system realtime. This can be used for training purposes in work that require for instance lifting to aid the person performing the work to develop correct lifting techniques, by reminding the person in the situation if something is done incorrectly

### Example III

### Sport

In this embodiment the system is used to monitor sport activities and to assist training in sport situations. The sensors are attached before the sport activity and the device can provide realtime feedback during the exercises (like in the rehabilitation example), and statistics are gathered to show the progress of the training. In a professional sport setting the web interface may be monitored by a personal trainer, physiotherapist or similar. In an amateur or leisure setting the training results will be monitored by the athlete himself/herself. Similar methods used in the gait training can be employed to for example running, which again can be used both in a professional or amateur setting.

### Example IV

### Integration with other sensors

The infrastructure of the proposed system makes it possible to replace or supplement the motion sensors with other types of health sensors via for example Bluetooth.

For example an elastic band sensor can be used with the system. This sensor makes it possible to monitor the force on the elastic band during training. The proposed system can receive data from the sensor and thus count repetitions of training and monitor the force exercised on the elastic. As with the motion sensor setup the exercise data is stored as statistics on the device and is also uploaded to the server. The physiotherapists use the same web interface to plan and monitor the training. Specific exercises targeted to the specific sensor are created with the exercise editor. It is thus the exercise that defines which sensors that are needed to perform the exercise, and this makes the system flexible in terms of combining sensors and mixing exercises.

Another example is that the system can be used with a pneumatic pressure sensor integrated in a positive expiratory pressure (PEP) device that can be used by patients with chronic obstructive pulmonary disease (COPD). As in the example above, special exercises are created for the PEP device, and the therapists plan and monitor the training with the same web interface.

The system can also be used with an optic sensor such as a camera (for example a web camera or built in camera) or a camera with additional depth information (for example the Microsoft Kinect). Using computer vision the camera can provide motion data for the system for example in the form of skeleton tracking.

1.-14. (canceled)

15. A method for analysing movements of main body parts of a moving person, the method comprising the steps of
attaching one or more sensors to selected main body parts, each sensor comprising means for wireless communication of data,
calibrating data from the one or more sensors, and
mapping the calibrated sensor data onto a virtual 3D avatar.

16. A method according to claim 15, wherein the mapping of the calibrated sensor data is performed real time.

17. A method according to claim 15, further comprising the step of making the calibrated sensor data available via a web service.

18. A method according to claim 15, wherein the step of calibrating data involves position calibration, calibration via exercise, dynamic calibration and/or hands free calibration via automated position calibration.

19. A method according to claim 15, further comprising the step of using real time audio feedback, said audio feedback being based on performance, character and/or quality of exercises and/or movements.

20. A method according to claim 15, further comprising to step of extracting algorithms for use in developing additional exercises.

21. A method according to claim 15, wherein the moving person is a patient undergoing rehabilitation.

22. Use of the method according to claim 15, said use comprising creating and editing training exercises via an exercise editor.

23. Use of the method according to claim 15, wherein the moving person is a patient undergoing rehabilitation.

24. A mobile system for analysing movements of main body parts of a moving person, the mobile system comprising
one or more sensors adapted to be attached to selected main body parts of the moving person, each sensor comprising means for wireless communication of data,
processor means for calibrating data from the one or more sensors, and
means for mapping the calibrated sensor data to a virtual 3D avatar.

25. A mobile system according to claim 24, further comprising an accessible unit for hosting at least the calibrated sensor data.

26. A mobile system according to claim 25, wherein the accessible unit is adapted to communicate with a web service.

27. A mobile system according to claim 24, wherein a number of the one or more sensors is/are adapted to host at least the calibrated sensor data.

28. A mobile system according to claim 27, wherein the calibrated sensor data are hosted on a SD card.

29. A mobile system according to claim 24, wherein the processor means form part of a portable device, such as a mobile phone or a tablet.

30. Use of a mobile system according to claim 24, wherein the moving person is a patient undergoing rehabilitation.

31. A computer program product for performing the method according to claim 15, when said computer program product is run on a processor, such as a computer.
